(19) United States
(12) Patent Application Publication
(10) Pub. No.: US 2009/0103047 A1
(43) Pub. Date: Apr. 23, 2009

(54) Tonometer Using Camera and Ambient Light

(75) Inventors: Francis Y. Falck, JR., Stonington, CT (US); Robert W. Falck, Pawcatuck, CT (US)

Correspondence Address: BROWN & MICHAELS, PC;400 M & T BANK BUILDING, 118 NORTH TIOGA ST, ITHACA, NY, 14850, US

(73) Assignee: FALCK MEDICAL, INC., Mystic, CT (US)

(21) Appl. No.: 12/254,509

(22) Filed: Oct. 20, 2008

Related U.S. Application Data
(60) Provisional application No. 60/981,930, filed on Oct. 23, 2007.

(51) Int. Cl. A61B 3/10

(52) U.S. Cl. 351/205

(57) ABSTRACT

A tonometer uses ambient light available in an eye examining room, rather than a dedicated source of light, to examine a characteristic of an eye. A digital camera in the tonometer views an image of the eye as it is engaged by a contactor that applanates or indents the cornea. An electromagnetic mount for the contactor can supply a force pressing the contactor against the eye. While the examiner observes the resulting image a strain gauge can also measure the deformation pressure applied to the eye by the contactor. A microprocessor can then determine a characteristic of the eye from signals supplied by the camera and the strain gauge or the electromagnet force applier.

### REFERENCE TO RELATED APPLICATIONS

This application claims benefit under 35 USC §119(e) of subject matter disclosed in Provisional Application No. 60/981,930, filed 23 Oct. 2007, entitled “Tonometer Using Camera and Ambient Light”.

### FIELD OF THE INVENTION

Tonometry of the eye

### BACKGROUND

Tonometers measure intraocular pressure (IOP) of an eye. A preferred form of tonometer applanates or indents an area of the cornea and uses a light source and a detecting system to determine the size of the corneal area that is deformed by the contact and the force involved in pressing a contactor against the cornea. The tonometer can then determine IOP from the relationship between the force applied and the size of the corneal area that is deformed. Pertinent examples of such tonometers include U.S. Pat. Nos. 6,179,779, 6,736,778, and 7,153,267 to Falck; Publication No. 20030236470 to Falck; U.S. Pat. No. 5,190,042 to Hock; U.S. Pat. No. 6,083,160 to Lipman; U.S. Pat. No. 5,671,737 to Harosi; and U.S. Pat. No. 6,776,756 to Feldon et al.

This invention improves on previous tonometers in several ways. These include simplifying optical systems, force measurement, and detection systems, and eliminating the need for a dedicated light source. The goals are a tonometer that is accurate, safe, versatile, robust, and inexpensive.

### SUMMARY

The tonometer of this invention uses a digital camera to observe a deformed corneal area so that the camera can determine the size of the deformation from the observed image. We have found that this can be done using ambient light, rather than requiring a dedicated source of illumination. The invention also includes a simple and effective way of mounting a force responsive contactor and of measuring a force used in pressing the contactor against a cornea to secure an IOP measurement.

### DRAWINGS

FIG. 1 is a partially schematic, cross-sectional view of a preferred embodiment of the inventive tonometer.

FIG. 2 is a view of a contactor showing a deformed corneal area.

FIG. 3 is a view of the contactor of FIGS. 1 and 2 from the eye being examined.

FIG. 4 is a schematic, side view of another form of tonometer provided with a beam splitter for use on a slit lamp microscope.

FIG. 5 is a schematic view similar to the view of FIG. 1 showing a manual operation system for pressing a contactor against a cornea.

FIG. 6 is a schematic view of another form of tonometer that requires replacement of used contactors.

### DETAILED DESCRIPTION

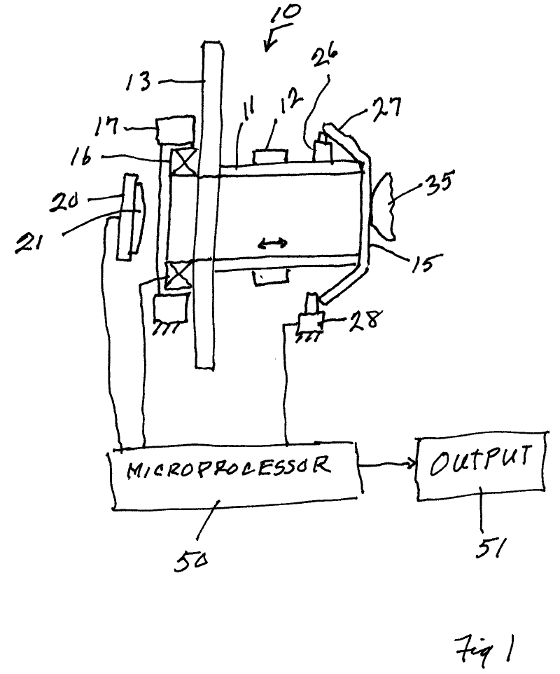

Tonometer 10, as schematically illustrated in FIG. 1, includes a tube or other structure 11 that is movable as indicated by the double-headed arrow, to press a contacting surface or window 15 against a cornea of an eye 35. The movement required is about 1 mm, but is not necessarily limited to that amount. A preferably digital camera 20 is positioned to observe the size of a corneal area that is applanated or indented by contactor surface 15. A linear bearing 12 supports tube 11 for the required axial movement, and a resilient element 13 holds tube 11 and accommodates the axial movement while supplying a small resistance to the movement. Element 13 can advantageously be formed as an audio speaker diaphragm, which is readily available and well understood. Other resilient elements and supports for contactor 15 are also possible.

The embodiment of FIG. 1 includes a force generating system using an annular coil 16 secured to diaphragm or resilient element 13, and a fixed annular magnet 17 surrounding coil 16. A current applied to coil 16 can then move tube 11 to press contactor 15 against a cornea, and stopping the current to coil 16 can allow tube 11 to retract from a cornea under the resilient influence of element 13.

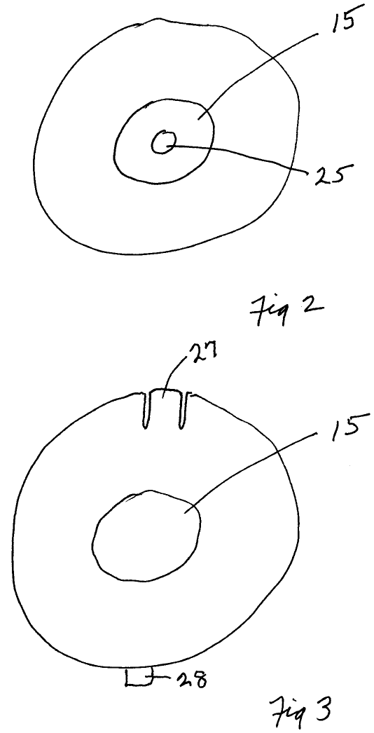

Tonometer 10 uses ambient illumination such as is generally available in places where eyes are examined. We have found that ambient light in an examining room is adequate to provide camera 20 with a view of the size of a deformed area 25 of a cornea against which surface 15 is pressed. This simplifies the construction of tonometer 10 by eliminating the need for a dedicated light source. In effect, camera 20 observes an eye as contactor 15 approaches. Then when surface 15 contacts a cornea, a small deformation area 25 occurs, as shown in FIG. 2. This area 25 can be enlarged by pressing contactor 15 with increasing force against the cornea.

The deformation of a cornea by contactor 15 can cause applanation or indentation of the cornea. Either of these slightly decreases the volume of the eye and raises the eye pressure. The applanated or indented area of the cornea is observable as an image viewed by camera 20, which can see from the image the extent of the applanation or indentation. A schematically shown lens 21 can facilitate the viewing by camera 20.

A signal from camera 20 can determine the size of applanated or deformed area 25 in various geometrical ways. These can be based on the fact that some of the pixels in camera 20 receive significantly reduced illumination in the observed image area 25, so that the difference between well illuminated pixels and reduced illumination pixels can be exploited. Diameters of the deformed area 25 can be used to calculate the size of area 25, and counting the illuminated or unilluminated pixels can also produce a deformed area determination.

When coil 16 and magnet 17 are used to apply force to press contactor 15 against a cornea and enlarge an affected area 25, then the current supplied to coil 16 can also produce a measure of the force applied in pressing contactor 15 against the cornea. The force applied as evidenced by the current to coil 16 and the size of the area affected, as evidenced by an image signal from camera 20, can then indicate IOP. This is preferably done with microprocessor 50 which can operate coil 16, collect signals from camera 20, coil 16, and strain gauge 28, and produce an output 51 indicating a characteristic of the eye being examined. Such a characteristic can include intraocular pressure, ocular pulse pressure, ocular blood flow, and tonography.

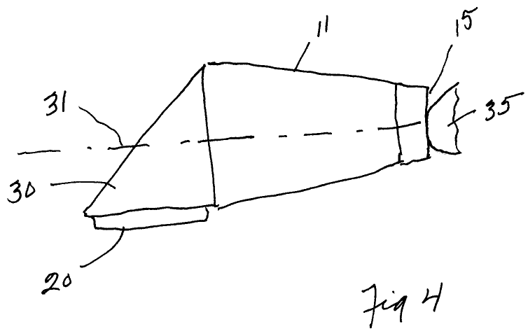

Another way of determining the force applied in pressing contactor 15 against a cornea is by use of strain gauge 28 as shown in FIGS. 1, 3 and 5. This can be positioned to sense movement of tube 11 or movement of contactor 15 against the resilient bias of element 13. Alternatively, the amount of current applied to coil 16 can also measure force applied to contactor 15, and the two different force measurements can be used corroboratively: one being the force derived from the current applied to coil 16 to move diaphragm 13, and the other being movement detected by strain gauge 28. Each of these can represent force applied to applanator window 15.

Contactor 15 is preferably molded of resin material that is thin, clear, and flat in a central surface area 15. Window 15 and the other elements of movable tube 11 are preferably made compact and lightweight to simplify the support and movement operations and improve measurement accuracy. Shapes other than tubes and flat windows can also work.

It is generally preferred for tonometers that an element contacting the cornea be disposable to prevent transfer of microorganisms or prions from one eye to another. For this purpose, contactor 15 is preferably required to be replaced after examining a pair of eyes. This can be done by using strain gauge 26, which is deflected when contactor 15 is pressed into an operating position. A flexible region 27 of contactor 15 moves strain gauge 26 as contactor 15 is mounted on tonometer 10. The flexible portion 27 of contactor 15 is preferably configured so that strain gauge 26 can distinguish between a used or previously mounted contactor and a new or not previously mounted contactor. There are many ways that this can be done, and these include forming contactor 15 with a flexible tab 27 that engages a strain gauge 26 either from direct axial pressure, or from rotational movement that may be required to seat contactor 15 in place. Tonometer 10 can be made inoperable until a fresh contactor 15 is properly positioned, and strain gauge 26 can determine this and also distinguish between a used contactor that is reinserted and an unused contactor inserted for the first time.

The embodiment of FIG. 6 illustrates an alternative possibility. Its contactor 15 is thimble shaped with a side wall formed to include a flexible tab 27. Contactor 15 preferably has a snap fit onto the end of tube 11, which is mounted on diaphragm 13. Snapping contactor 15 onto the end of tube 11 requires that strain gauge 26 measure the required flexure of element 27. If contactor 15 was previously mounted on the tonometer, element 27 will flex more easily than if contactor 15 is mounted for the first time on tube 11. This allows strain gauge 26 to distinguish between a previously used contactor and a previously unused contactor.

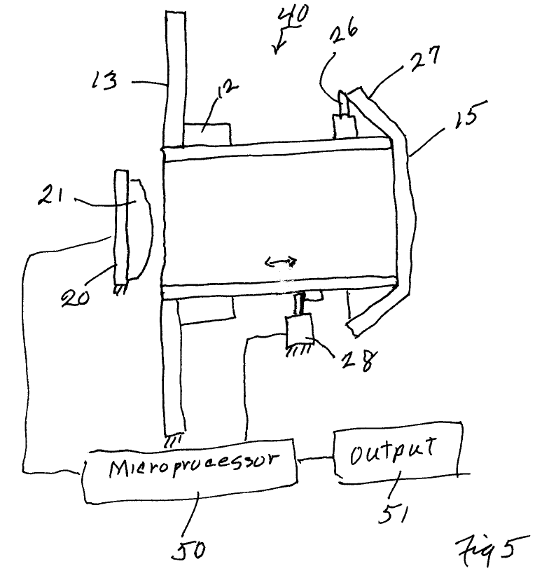

Since many tonometers are mounted on slit lamp microscopes where they enable an operator to view the eye and the affected corneal area during an examination, tonometer 10 can also accomplish this. As schematically shown in FIG. 4, a beam splitter 30 can direct light to a camera 20 positioned along side an optical viewing axis 31. An operator looking through beam splitter 30, along optical access 31, can observe an eye 35 being examined, while camera 20 can also observe via the beam splitter 30 the size of an area deformed by contactor 15.

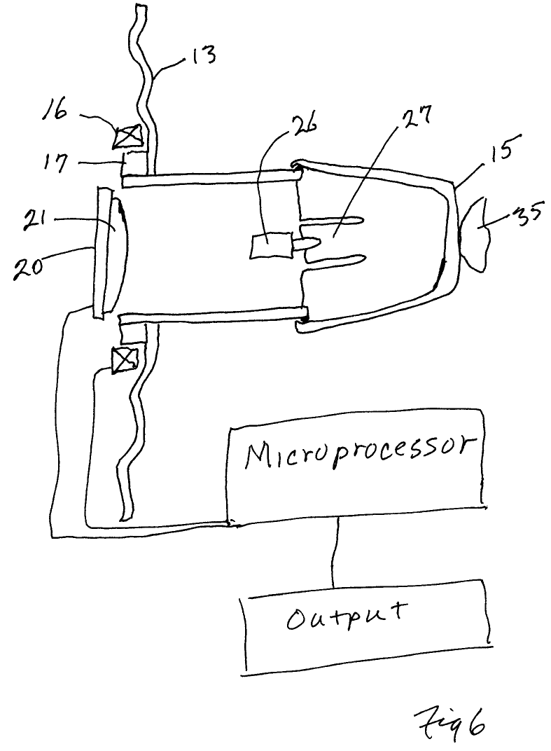

Tonometer 40, as schematically shown in FIG. 5, eliminates coil 16 and magnet 17 and relies on manual force to press contactor 15 against a cornea. A resilient support 13, such as an audio speaker diaphragm, resiliently holds tube 11 and allows for its movement as indicated by the double headed arrow. The amount of force manually applied is preferably monitored by strain gauge 28, which can measure the displacement of tube 11 and window 15. Linear bearing 12 supports tube 11 for this motion. A manually forced contactor 15, such as illustrated in FIG. 5, can also be provided with a beam splitter 30 to move camera 20 off a viewing axis along which an operator can observe. Also, a manually pressed contactor can be used in either a portable or slit lamp mounted tonometer. Microprocessor 50 can receive signals from camera 20 and strain gauge 28 and can then calculate a characteristic of the eye being examined and provide the calculation to an operator via output 51.

Another difference in the tonometer of FIG. 6 is that permanent magnet 17 is formed as part of tube 11. Coil 16 and magnet 17 are preferably part of a miniature audio speaker, which is compact, inexpensive, and readily available. The embodiment of FIG. 6 also eliminates any strain gauge measuring movement of contactor 15. Such a strain gauge 28, as shown in FIGS. 1 and 5 is necessary if the contactor pressing force is applied manually, but is an optional possibility when contactor pressing force is applied by coil 16 and magnet 17.

What is claimed is:

1. A tonometer using a corneal contactor arranged to be pressed with variable force against a cornea of an eye to be examined, the tonometer comprising:
a camera arranged to view ambient light reflected from the eye to reveal a deformation of the cornea that is caused by the contactor engaging the cornea;
a determiner of the variable force applied in pressing the contactor against the cornea; and
a microprocessor arranged to receive a signal from the camera representing a size of the corneal deformation and a signal from the determiner representing the variable force so that the microprocessor calculates a characteristic of the eye being examined.

2. The tonometer of claim 1 wherein the determiner is a strain gauge.

3. The tonometer of claim 1 wherein the determiner is a solenoid.

4. The tonometer of claim 1 wherein the contactor is mounted on a miniature audio speaker.

5. The tonometer of claim 1 wherein the variable force is applied by a solenoid.

6. The tonometer of claim 1 wherein an element of the contactor is flexed as the contactor is mounted on the tonometer, and a strain gauge on the tonometer measures the flexure of the contactor element to ensure that a fresh contactor is used for each examination of a pair of eyes.

7. In a tonometer having a corneal contactor arranged to be pressed with a variable force against the cornea of an eye being examined, and a detector of light representing a variable size of a corneal area deformed by the contactor, the improvement comprising:
the detector is a digital camera viewing the cornea;
the light representing the deformed corneal area is reflected ambient light; and
an indicator is arranged to determine a force employed in pressing the contactor against the cornea.

8. The tonometer of claim 7 wherein a solenoid applies the variable force.

9. The tonometer of claim 7 wherein the contactor is mounted on a miniature audio speaker.

10. The tonometer of claim 7 including a microprocessor arranged to determine a characteristic of the eye based on input from the detector and the indicator.

11. The tonometer of claim 7 wherein the indicator is a strain gauge.

12. The tonometer of claim 7 wherein the indicator is a solenoid arranged to apply the variable force.

13. The tonometer of claim 7 wherein the tonometer has a strain gauge positioned to measure flexure of an element of the contactor as the contactor is mounted on the tonometer.

14. A method of measuring a characteristic of an eye by using a tonometer, the method comprising:
illuminating the eye to be examined with ambient light;
pressing a contactor against the cornea of the eye with a variable force;
measuring the variable force;
viewing with a digital camera a reflected ambient light image of a corneal area deformed by the contactor; and
using a microprocessor to receive a signal from the camera and a signal from the force measurer to determine the characteristic of the eye.

15. The method of claim 14 including using a strain gauge to measure the variable force.

16. The method of claim 14 including measuring the variable force with a solenoid that applies the force to press the applanator against the cornea.

17. The method of claim 14 including measuring flexure of an element of the contactor as the contactor is mounted on the tonometer to verify that the contactor has not been previously used.
